(19) United States
(12) Patent Application Publication
(10) Pub. No.: US 2011/0201888 A1
(43) Pub. Date: Aug. 18, 2011

(54) Medical Devices and Methods

(76) Inventor: Sarah N. Verner, Sterling Heights, MI (US)

(21) Appl. No.: 13/030,378

(22) Filed: Feb. 18, 2011

Related U.S. Application Data
(60) Provisional application No. 61/305,649, filed on Feb. 18, 2010.

(51) Int. Cl. A61B 1/06

(52) U.S. Cl. 600/160

(57) ABSTRACT

The present invention generally provides methods and devices for removing fluid from a medical device, such as a access device having a viewing port. The viewing port may comprise a lens formed of a generally transparent piezoelectric material, and one or more electrodes may be operatively associated with the lens to cause the lens to vibrate in a desired mode of vibration, such as to shake off and/or atomize fluid or other debris on the lens.

### RELATED APPLICATIONS

This application claims the benefit of U.S. Provisional Application 61/305,649 filed Feb. 18, 2010.

### FIELD OF THE INVENTION

The present invention relates to methods and devices useful with respect to medical procedures, such as methods and devices for maintaining visibility during surgical procedures, including methods and devices employing piezoelectric materials, such as in connection with portions of medical devices through which procedures are viewed and/or through which portions of the body are accessed.

### BACKGROUND OF THE INVENTION

During endoscopic surgery, an endoscope may be directed through passageway, such as through a naturally occurring body orifice. The endoscope may include one or more view ports (such as a camera lens or viewing lens), and it may be desirable to maintain the view port clean and generally free of materials that could otherwise obscure the portion of the body or the procedure being viewed through the endoscope.

During laparoscopic surgery, one or more small incisions are formed in the abdomen and a trocar is inserted through the incision to form a pathway that provides access to the abdominal cavity. The trocar is used to introduce various instruments and tools into the abdominal cavity, as well as to provide insufflation to elevate the abdominal wall above the organs. During such procedures, a scoping device, such as a relatively small endoscope or laparoscope, is inserted through one of the trocars to allow a surgeon to view the operative field on an external monitor coupled to the scoping device.

Scoping devices are often inserted and removed through a trocar multiple times during a single surgical procedure, and during each insertion and each removal they can encounter fluid that can adhere to the scopes lens and fully or partially impede visibility through the lens. Furthermore, a scope can draw fluid from inside or outside a patients body into the trocar, where the fluid can be deposited within the trocar until the scope or other instrument is reinserted through the trocar. Upon reinsertion, fluid can adhere to the scopes lens. The scopes lens thus needs to be cleaned to restore visibility, often multiple times during a single surgical procedure. With limited access to a scope in a body, each lens cleaning can require removing the scope from the body, cleaning the scope lens of fluid, and reintroducing the scope into the body. Such lens cleaning is a time-consuming procedure.

US 2008/0081948 published Apr. 3, 2008 “Apparatus for Keeping Clean a Distal Scope end of a Medical Viewing Scope” is incorporated herein by reference in its entirety, and discloses a device including a tube, an annular sheath, and a handpiece. The tube has a proximal end fluidly connectable to irrigation fluid. US 2009/0234193 published Sep. 17, 2009, “Apparatus for Cleaning a Distal Scope end of a Medical View Scope” is also incorporated herein by reference in its entirety.

US Patent Application 2009/0281478 published Nov. 12, 2009, “Vibratory Trocar” is incorporated herein by reference in its entirety, and discloses devices and methods for removing fluid from a trocar.

U.S. patent application Ser. No. 12/110,724, filed on Apr. 28, 2008 and entitled “Absorbing Fluids in a Surgical Access Device;” U.S. patent application Ser. No. 12/110,727, filed on Apr. 28, 2008 and entitled “Scraping Fluid Removal in a Surgical Access Device”; U.S. patent application Ser. No. 12/110,742, filed on Apr. 28, 2008 and entitled “Wicking Fluid Management in a Surgical Access Device”; and U.S. patent application Ser. No. 12/110,755, filed on Apr. 28, 2008 and entitled “Fluid Removal in a Surgical Access Device”, all of which are hereby incorporated by reference in their entireties, disclose one or more devices and/or methods useful in addressing scraping, absorbing, or otherwise removing material that is not desired from an access device.

Still, scientists and engineers continue to search for new devices and methods for use in medical devices and procedures, such as can be helpful for maintaining or restoring visibility through a lens or other viewing port during a surgical procedure.

### BRIEF DESCRIPTION OF THE DRAWINGS

The invention will be more fully understood from the following detailed description taken in conjunction with the accompanying drawings, in which:

FIG. 1 is a schematic illustration of a piezoelectric lens and signal generator.

FIG. 2 illustrates a top view of the lens assembly of FIG. 1.

FIG. 3 illustrates a displacement/vibratory mode of the lens of FIG. 1.

FIG. 4 is a schematic illustration of a piezoelectric lens assembly.

FIG. 5 illustrates a cross section of the piezoelectric lens having ring shaped electrodes disposed on top and bottom surfaces of the lens.

FIG. 6 illustrates a displacement/vibratory mode of the lens of FIG. 5.

FIG. 7 is a schematic illustration of a piezoelectric lens assembly.

FIG. 8 is a schematic illustration of lens assembly having a lens and associated ring assembly comprising multiple piezoelectric elements.

FIG. 9 is an enlarged view of the ring assembly of FIG. 8.

FIG. 10 illustrates a sheath, such as sheath sized to receive an endoscope, the sheath comprising conductive spring elements along its length and a piezoelectric lens assembly disposed at the distal end of the sheath.

FIG. 11 illustrates an viewing scope, sleeve, and piezoelectric lens assembly associated with the distal end of the sleeve.

FIG. 11A illustrates a enlarged view of the lens assembly of FIG. 11 and illustrating a snap feature of a cap of the piezoelectric lens assembly.

FIG. 12 illustrates a trocar comprising a piezoelectric lens assembly disposed within the trocar.

FIG. 12A illustrates an enlarged portion of the trocar of FIG. 12 and illustrating the piezoelectric lens assembly in a closed configuration with respect to an instrument channel of the trocar.

FIG. 12B illustrates an enlarged portion of the trocar of FIG. 12 and illustrating the piezoelectric lens assembly in an open configuration with respect to an instrument channel of the trocar.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for maintaining or restoring visibility through a viewing port of a device during surgical procedures.

A person skilled in the art will appreciate that the term fluid as used herein is intended to include any substance that, when on a surgical instrument, can adversely affect the functioning of the instrument or a surgeon's ability to use it. Fluids include any kind of bodily fluid, such as blood, and any kind of fluid introduced during a surgical procedure, such as saline. Fluids also include fluid/solid mixtures or fluids with particles (such as pieces of tissue) suspended or located therein, as well as viscous materials and gases. A person skilled in the art will also appreciate that the various concepts disclosed herein can be used with various surgical instruments during various procedures, but in certain exemplary embodiments the present invention is particularly useful during endoscopic and/or laparoscope procedures, and more particularly during procedures in which a device, such as an laparoscope or endoscope, is passed into the body, such as through a naturally occurring orifice or through a surgical access device, such as a trocar.

In one embodiment, a viewing port of a medical device may comprise a piezoelectric element, such as a generally transparent lens formed of a piezoelectric material. One suitable piezoelectric material is quartz.

The piezoelectric lens may be configured to provide vibration in one or more directions and/or one or more modes of vibration. For instance, in one embodiment the piezoelectric lens may be configured to provide radial or horizontal vibration of the lens (vibration in the plane of the lens) when the piezoelectric material of the lens is electrically activated. Radial or horizontal vibration of the lens may be useful to shake off or otherwise displace fluids, solids, or combinations of fluids or solid, such as body fluids that contact or adhere to the lens during insertion into the body, or withdrawal from the body, of the device that incorporates the lens.

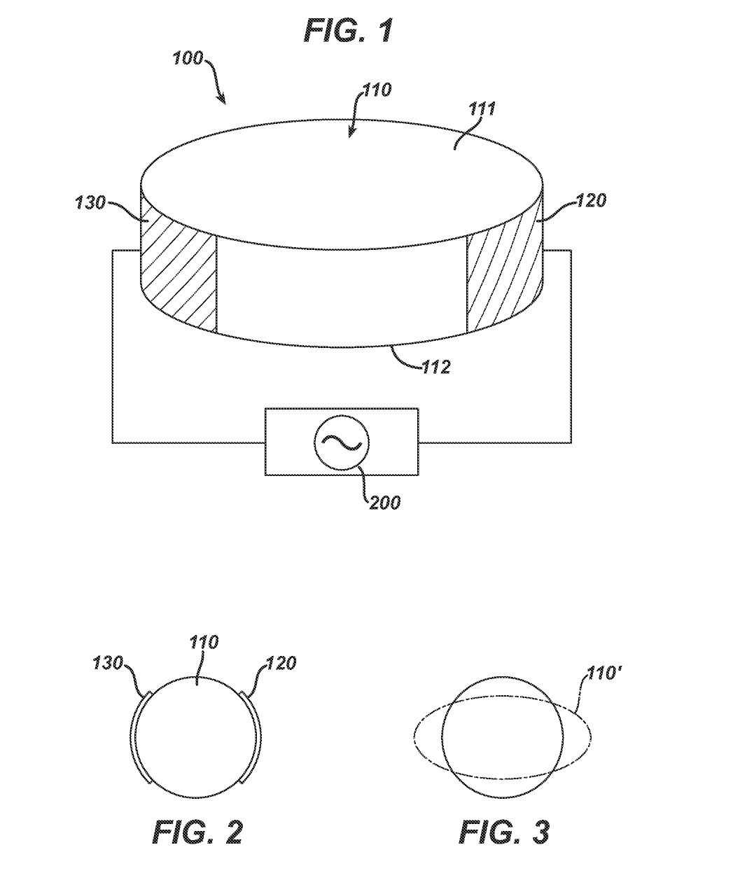

FIG. 1 provides a schematic illustration of a viewing device 100 comprising a generally transparent lens 110 comprising a piezoelectric material, and two electrodes 120 and 130 joined to circumferentially spaced apart portions of the lens, such as at circumferentially opposed edges of the lens 110. The lens 110 can be formed of a generally transparent piezoelectric material such as quartz. The lens can have a top, proximal (toward viewer's eye) surface 111, and a lower, distal (facing away from viewer's eye) surface 112. The surfaces 111 and 112 are shown as generally flat planar surfaces of illustration purposes, but it will be understood that the lens can be provided to have planar/convex, planar/concave, convex/planar, concave/planar, concave/convex, convex/concave, convex/convex, concave/concave, or any other suitable lens configurations.

A power source 200 may be employed to provide AC electric power to the electrodes 120 and 130 at a suitable frequency or combination of frequencies to excite the lens 110 to vibrate the lens, such as in the plane of the lens. For instance, in one or more applications a frequency in the 50-500 kHz range may be desired. In one embodiment, a bridge rectifier, such as a full wave bridge wave rectifier may be employed, including a Wheatstone bridge with the resistors replaced with diodes. The benefit of using a bridge rectifier is conversion of an AC input into a DC output. In addition, the bridge rectifier can be employed to provide an out frequency double that of the input frequency. Accordingly, in some applications it may be desirable to provide a 25-250 kHz AC input to the bridge rectifier to provide a 50-500 kHz output signal to the piezoelectric element/lens. For instance, in one embodiment a generally sinusoidal AC input can be provided as input to the bridge rectifier. In other embodiments, other suitable wave forms, such as square wave may be employed.

FIG. 2 illustrates a top view of the lens 110 with the power source turned off or otherwise disconnected from the electrodes, and FIG. 3 illustrates (in phantom lines) the horizontal mode of vibration of the lens indicated as 110′F. The vibration mode of the lens depicted in phantom in FIG. 3 is for illustration purposes only, and will be understood that various other modes or combination of modes of vibration of the lens may be employed. Additionally, if desired, two or more pairs of electrodes may be employed, such as at spaced apart and generally opposed positions around the circumference of the lens.

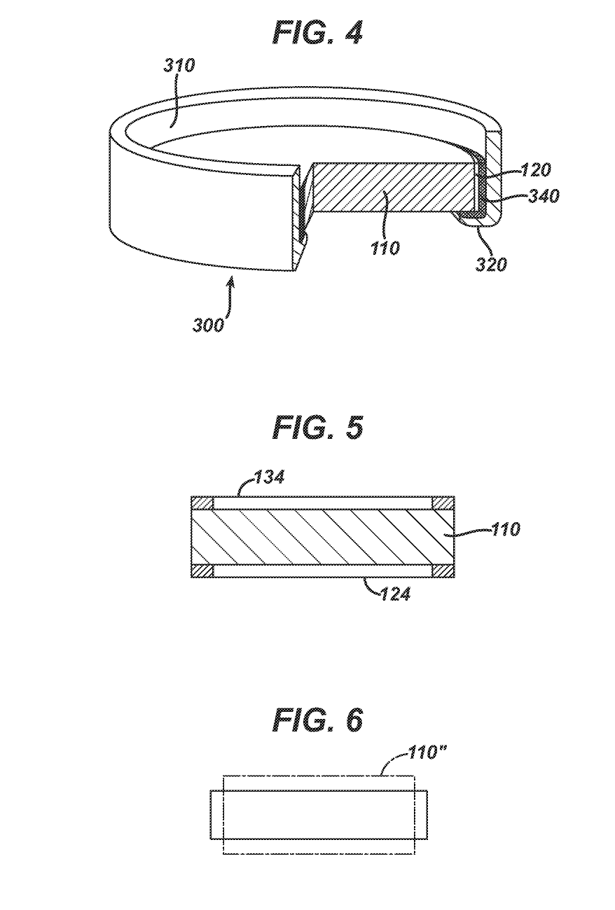

FIG. 4 illustrates one embodiment of assembly comprising the viewing device 100 of FIG. 1. The assembly is shown including a housing which may be in the form of an annular cap 300 having a generally ring shaped body 310 and a support ledge 320 extending radially inwardly from a distal end of the body 310. In FIG. 4, a portion of the cap 300 and the lens 110 is shown cut away to illustrate cross-sectional features of the assembly.

In the embodiment shown in FIG. 4, the cap 300 supports lens 110 with a layer 340 disposed between the lens 110 and the cap 300. The layer 340 may be provided to isolate or otherwise space a portion of the lens 110 from the cap 300, and to protect the piezoelectric material from fracturing against the cap 300. For instance, if the cap 300 is formed of a generally stiff or rigid plastic or polymeric material, the layer 340 may comprise a relatively softer material, such as a damping material to assist in protecting the lens 110 from fracturing against cap 300. For instance, the layer 340 may comprise a layer of rubber, of an elastomeric material, and/or a visco-elastic material. The layer may have a hardness less than that of the material from which the cap is formed. Alternatively, the cap 300 may be formed from, or incorporate a material that provides damping.

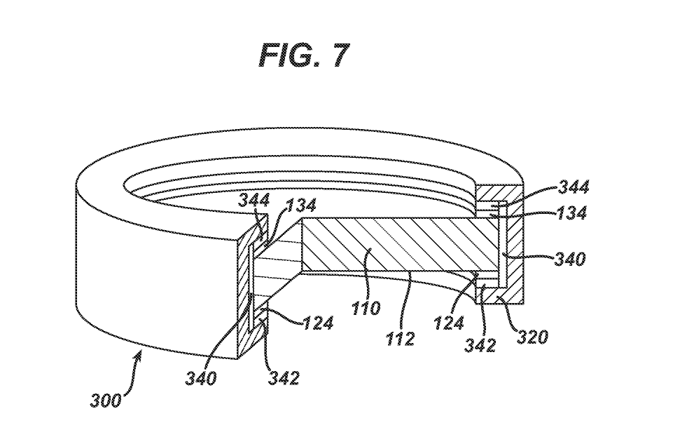

FIGS. 5-7 are directed to an assembly for providing vertical vibration. FIG. 5 is a cross-sectional schematic illustration of a piezoelectric lens 110 and generally ring shaped annular electrodes 124 and 134. The electrode 124 is shown disposed on a bottom surface of the lens 110, and electrode 134 is shown disposed on a top surface of the lens 110. FIG. 6 illustrates in phantom a vertical vibration mode indicated as 110″ when the electrodes 124/134 are energized (e.g. up and down in a direction along the axis of viewing through the lens 110). FIG. 7 illustrates a partially cutaway view of the lens 110 and electrodes 124/134 supported in a housing, such as cap 300.

In FIG. 7, the assembly is shown including a first damping ring 342 disposed between the ring electrode 124 and a lower cap ledge 320, and a second damping ring 344 disposed between the ring electrode 134 and upper cap ledge of cap 300. The assembly can also include a layer 340 (such as formed of damping material) disposed circumferentially outward of the lens 110, such as between the circumferential edge of the lens 110 and the cap 300.

The ring electrodes 124 and 134 disposed on opposed faces of the lens 110 may be employed to provide vertical vibration of the lens 110 (vibration generally parallel to or aligned with the viewing axis through the lens). Such vertical vibration may be employed to atomize or otherwise break-up or displace debris (solid or liquid) deposited on the end of the lens 110.

For instance, but without limitation, it can be desirable to oscillate the lens 110 at the same or substantially the same frequency as the natural frequency of the debris on the lens 110. This natural frequency can have a dependence not only on the mass and properties of the lens 110, but also on the mass and material composition and properties of the debris. In one embodiment, the medical device can include a controller which employs a “roaming” frequency generator for energizing the electrodes 124 and 134.

By way of example, the lens 110 having the debris on it's surface (e.g. lower surface 112 in FIG. 7) may be energized at a first driving frequency (e.g. F1) to provide forced vibration of the lens/debris combined mass at frequency F1. The electrical current can then be turned off, allowing the lens (and attached debris) to continue to vibrate in a generally free, unforced manner. The lens 110 (with debris) will continue to vibrate for a short period of time after the power is turned off, and because the lens 110 is formed of a piezoelectric material, an AC output voltage having a frequency (e.g. F2) will be generated. Generally, frequency F2 will be different from frequency F1. This output voltage and frequency F2 can be recorded using a microprocessor associated with the signal generator. The frequency F2 will be (or will be representative of) the natural vibratory frequency of the combined lens/debris vibratory system. The microprocessor can be programmed to next apply a driving electrical current at the natural frequency (e.g. F2) to this recorded frequency F2 to the electrodes 124 and 134. By energizing the electrodes at a frequency characteristic of the natural frequency of the combined lens/debris mass system, the debris will be more effectively atomized and/or removed.

The steps noted above can be repeated multiple times, so that as debris is atomized/removed (or as new debris becomes attached to the lens) the natural frequency of the lens/debris mass is tracked in real time, and the driving frequency is adjusted accordingly.

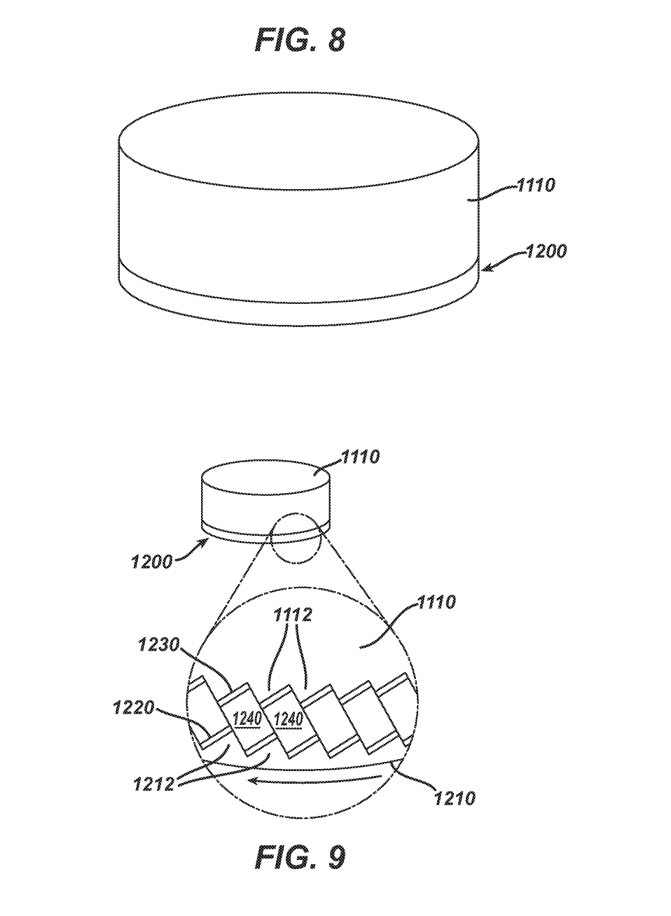

FIGS. 8 and 9 illustrate an embodiment which may be used to provide rotational motion. FIG. 8 provides a schematic illustration of a lens 1110 (which can be formed of any suitable material, and not necessarily a piezoelectric material) and an assembly 1200 operatively associated with at least one face or side of the lens 1110. Referring to FIG. 9, assembly 1200 is shown including a base ring 1210 having a plurality of inclined surface features, such as tooth shaped (or other suitably shaped) steps 1212 arranged circumferentially around the perimeter of base ring 1210. The lens 1110 is shown having a plurality of inclined surface features 1112 arranged circumferentially around the perimeter of lens 1110, and facing in a generally opposed manner with respect to surface features 1212.

As illustrated in FIG. 9, the assembly 1200 is shown including a plurality of piezoelectric elements 1240 with associated energizing electrodes 1220/1230. A piezoelectric element 1240 and associated electrode pair 1220/1230 is shown positioned between corresponding opposed features 1212 and 1112.

Each axis of vibration of the individual elements 1240 can be disposed at an angle with respect to the viewing axis through lens 1110. Power can be provided to the elements 1240 in a sequenced manner, such as in a circumferential direction in a one at a time fashion. By way of example, power can be sequenced from one electrode pair 1220/1230 to the next, around the circumference of the assembly 1200, to provide a circumferential motion or disturbance (for instance, somewhat like a crowd doing “the wave” around a circular stadium). The resulting circumferentially traveling strain/movement induced in the elements 1240 may be employed to cause the lens 1110 to move or shake (on a micro basis or macro basis) in a circumferential direction. The base 1210 can be positioned with respect to a medical viewing device, such as by being fixed to a distal end of a sheath, endoscope, trocar, or such as by being provided as a removable cap that can attached to and removed from a medical viewing device. A signal generator and controller can be employed to provide sequenced electrical power to the elements 1240 in the desired timing.

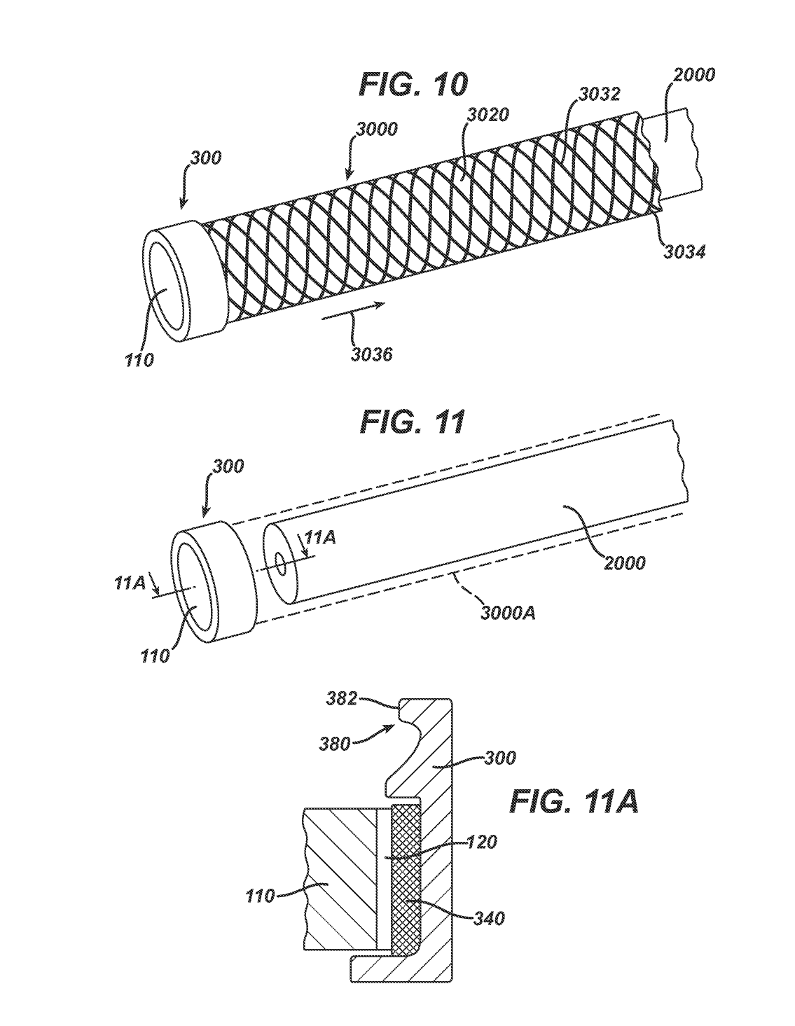

FIG. 10 illustrates an embodiment comprising a sleeve, such as a sleeve 3000 sized and shaped to receive an endoscope (labeled 2000) at least partially therethrough, and a cap 300 shown disposed at a distal end (working end disposed in the body) of sleeve 3000. The cap 300 can support a lens 110 formed of piezoelectric material. The sleeve 3000 may be formed of a flexible, pliable material having a conductor embedded therein or otherwise operatively associated with the sleeve. For instance, sleeve 3000 may comprise a flexible sheath 3020 formed of a suitable medical grade, biocompatible polymer, and one or more conductors (two conductors numbered 3032 and 3034 shown) which may be generally spiral or helical conductors along the length and about the circumference of the sleeve 3000.

The conductors may be embedded in the sheath 3020, joined to inner and/or outer surfaces of sheath 3020, or if sheath 3020 has a multiple layer construction, disposed between layers of sheath 3020. The conductors 3032/3034 may each have an insulating layer over a metallic core, and the conductors may provide electrical connection between a power source and electrodes associated with the lens 110. The conductors can be formed of a suitable conductive spring material, shape memory material, or the like, and the conductors 3032/3034 may be employed to provide radial compressive spring forces to hold the sheath 3020 snuggly against the endoscope 2000. The conductors 3032/3034 may also provide an axial biasing force (e.g. in the direction of arrow labeled 3036 in FIG. 10) to assist in holding the cap 300 against the distal end of the endoscope 2000, so that piezoelectric lens 110 is positioned firmly against the viewing port, lens, camera, and/or working channel at the distal end of the endoscope 2000.

Alternatively, referring to FIG. 11 and FIG. 11A, the piezoelectric lens 110 supported by cap 300 may be joined to a sleeve 3000A (shown in dotted line fashion) or the endoscope 2000 via a snap fit feature 380. The snap fit feature 380 may be in the form of a lip 382 formed on the proximal end of the cap 300, and the feature 380 may formed of a resilient material that allows the proximal end of the cap 300 to be snap fit onto the sheath or the scope 2000 itself.

FIG. 11A shows a partial cross-section of a cap 300 having snap fit feature 380. Alternatively, threaded fitting, bayonet fittings, or other attachment features may be employed to releasably join the cap 300 holding lens 110 to the distal end of a sheath, endoscope, or other viewing instrument. In yet other embodiments, the piezoelectric lens and associated electrodes may be a built in component of the endoscope 2000, such as where the piezoelectric lens provides part of the optics (such as a camera lens) of a medical instrument.

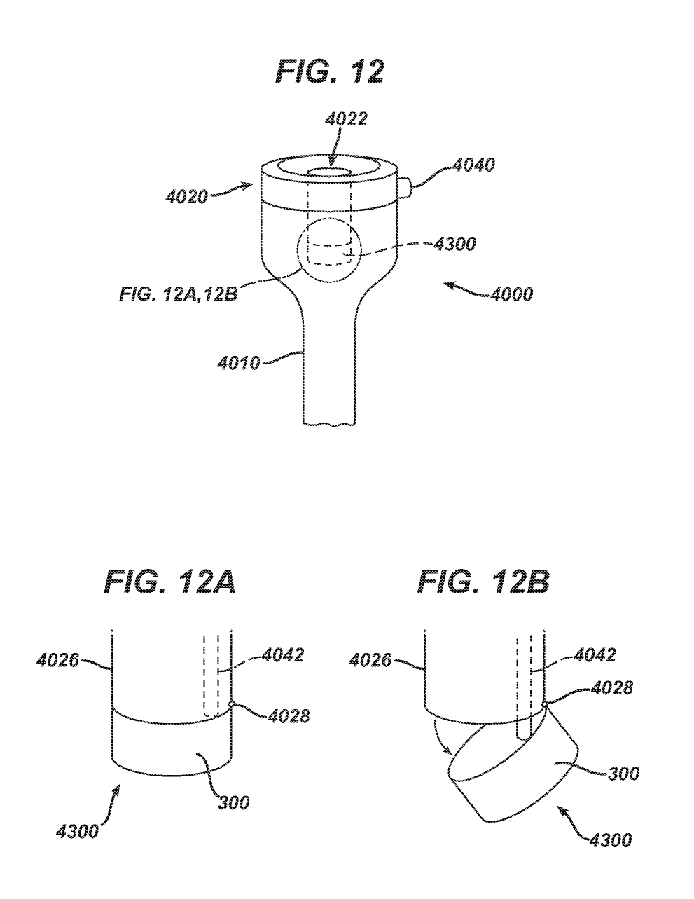

FIGS. 12, 12A and 12B illustrate a trocar 4000 incorporating a piezoelectric lens. Trocar 4000 may include a cannula 4010 sized and shaped to be inserted through the patients skin and into an operative space within the body. The trocar 4000 may also include a seal housing 4020, which may be releasably supported proximally of the cannula 4010. The seal housing 4020 may include one or more instrument openings (one shown in FIG. 12 and labeled 4022) through which laparascopic instruments may be inserted to extend through the cannula 4010 and into the interior of the patient's body.

One or more seals may be operatively associated with each instrument opening to prevent escape of insufflation gas. For instance, an instrument seal may be provided to provide sealing around the shaft of a laparascopic instrument when the instrument is inserted through the cannula 4010, and a zero closure seal (for instance a duckbill seal) may be employed to providing sealing when no instrument is inserted through the trocar.

As shown in FIGS. 12, 12A, and 12B, a piezoelectric assembly 4300 may be operatively associated with the instrument opening 4022, such as by being supported distally of the instrument opening 4022. The assembly 4300 may include a cap 300 and piezoelectric lens 110, as described previously.

In FIG. 12, a lead in passageway in the form of a tube 4026 extends distally from opening 4022. The assembly 4300 is shown disposed at the distal end of the tube 4026. The tube 4026 may be generally stiff, or alternatively, tube 4026 may be generally flexible and/or be pivotable with respect to the cannula 4010 and/or the housing 4020.

The cap 300 of assembly 4300 can be joined to the distal end of the tube 4026 so that the assembly is positionable in a closed position (FIG. 12A) and an open position (FIG. 12B). If desired, the assembly 4300 can provide a seal with respect to tube 4026, so that the assembly 4300 provides a seal to prevent escape of insufflation gases when no instrument is inserted through opening 4022.

In one embodiment, the cap 300 may be joined to the distal end of the tube 4026 by a hinge 4028. The hinge 4028 may be spring loaded (such as with a small torsion spring), such that assembly 4300 remains in the closed position unless and until a laparascopic instrument is inserted through opening 4022 and tube 4026 to push downward against assembly 4300, thereby causing assembly 4300 to pivot on hinge 4028 and thereby open the distal end of tube 4026 (pivoting shown in FIG. 12B). Alternatively, a manual opening feature such as push button 4040 may be provided, such as on the outside of the housing 4020, to open/pivot assembly 4300. The button 4040 may be used to actuate a push rod 4042 (shown in phantom in FIG. 12A) to cause the assembly 4300 to pivot as shown in FIG. 12B).

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination.

Preferably, the devices described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and its contents are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed:

1. A medical device comprising:
an access component having a proximal end and distal end;
a viewing port associated with the access component; and
a piezoelectric element operatively associated with the viewing port.

2. The medical device of claim 1 wherein the piezoelectric element comprises a lens.

3. The medical device of claim 1 wherein the piezoelectric element is operative to provide vibration of the element in the plane of the element.

4. The medical device of claim 1 wherein the piezoelectric element is operative to provide vibration of the element transverse to the plane of the element.

5. The medical device of claim 1 wherein the medical device comprises a plurality of piezoelectric elements.

6. The medical device of claim 1 wherein the access component comprises an endoscope.

7. The medical device of claim 1 wherein the access component comprises a trocar.

8. The medical device of claim 1 comprising an assembly associated with a distal end of the access device, and wherein the assembly comprises a cap, a piezoelectric lens supported by the cap, and at least two electrodes operatively associated with the piezoelectric element.

9. The medical device of claim 1 comprising a trocar having at least one instrument channel, and a piezoelectric lens assembly associated with the channel, wherein the lens assembly is movable from an open configuration to a closed configuration.

10. A method of operating a medical viewing device, the method comprising the steps of:
obtaining a medical viewing device having at least one viewing port;
vibrating the viewing port at a first driving frequency F1 with a driving signal;
measuring the free natural frequency of the viewing port upon termination of the driving signal and prior to decay of free vibration; and
vibrating the viewing port at a second driving frequency F2 corresponding to the measured natural frequency of the viewing port.

11. The method of claim 10 comprising vibrating the viewing port in the plane of the viewing port.

12. The method of claim 10 comprising vibrating the viewing port in a plane transverse to the viewing port.
